(19) United States
(12) Patent Application Publication
(10) Pub. No.: US 2023/0113060 A1
(43) Pub. Date: Apr. 13, 2023

(54) RADIOLOGY QUALITY DASHBOARD DATA ANALYSIS AND INSIGHT ENGINE

(71) Applicant: KONINKLIJKE PHILIPS N.V., EINDHOVEN (NL)

(72) Inventors: PRESCOTT PETER KLASSEN, CAMBRIDGE, MA (US); TIM PHILIPP HARDER, AHRENSBURG (DE); THOMAS BUELOW, GROSSHANSDORF (DE)

(21) Appl. No.: 17/913,252

(22) PCT Filed: Mar. 16, 2021
(86) PCT No.: PCT/EP2021/056589

§ 371 Date: Sep. 21, 2022

Related U.S. Application Data
(60) Provisional application No. 62/994,352, filed on Mar. 25, 2020.

(51) Int. Cl. G16H 40/20; G16H 30/20

(52) U.S. Cl. CPC G16H 40/20; G16H 30/20; G06F 3/0484

(57) ABSTRACT

A non-transitory computer readable medium (26) stores instructions readable and executable by at least one electronic processor (20) to provide statistical analysis on one or more radiology databases (30, 32) in conjunction with a workstation (18) having a display device (24) and at least one user input device (22). The instructions include: instructions readable and executable by the at least one electronic processor to provide a plurality of different analysis services (39) for selecting and processing exam data stored in the one or more radiology databases; and user guidance instructions readable and executable by the at least one electronic processor to guide a user in creating and executing a workflow (38) by providing a graphical user interface (GUI) (28) on the workstation having menus nd/or GUI dialogs (40) for user selection, configuration, and execution of an ordered sequence of analysis services and providing data persistence between the analysis services of the ordered sequence of analysis services.

### FIELD

The following relates generally to the radiology arts, radiology reading arts, radiology department performance assessment arts, radiology report quality assessment arts, and related arts.

### BACKGROUND

Radiology departments at hospitals or other large medical institutions perform a large number of imaging examinations (“exams”) targeting different anatomies (e.g. head, lungs, limbs, et cetera) for different purposes (oncology, cardiology, pulmonology, bone fracture assessment, et cetera), and using different imaging devices often of different imaging modalities such as magnetic resonance imaging (MRI), computed tomography (CT), positron emission tomography (PET), ultrasound, et cetera. A radiology department employs a staff of imaging technicians who operate the imaging devices, and a staff of radiologists who read the imaging examinations. In a large radiology department, there may be multiple work shifts so that the imaging devices are running over long time intervals (up to 24 hours per day in some cases), and likewise there may be multiple radiologist work shifts to read the large number of generated imaging examinations.

Assessing the work product of such a complex radiology department is difficult. Typically, some Key Performance Indicators (KPIs) are defined and tracked over time. Some examples of KPIs may include, by way of nonlimiting example: number of imaging examinations performed per day; radiologist read time per imaging examination; number of repeated imaging examinations; and so forth.

In many hospitals, such KPI analysis is delegated to quality managers or technologists who perform analysis tasks on top of their routine day-to-day responsibilities. Accordingly, very little time is available to dive into the data and gain a deeper understanding of patterns. Quality managers or technologists may have business education or training, especially in hospital management. In some cases, quality managers or technologists may have been promoted internally from the radiologist staff to radiology department management. Quality managers or technologists with such backgrounds usually have limited or nonexistent training in computer science and data analysis, and as such are not well-qualified to perform in-depth KPI analyses of the type needed to disambiguate between the various factors that potentially can adversely impact the radiology department work product. Such factors can include, but are not limited to, various combinations of: specific underperforming imaging technicians or radiologists; specific underperforming imaging devices; underperformance of specific imaging modalities; underperformance of specific work shifts; nonoptimal imaging examination workflows; examination scheduling issues; information technology (IT) system deficiencies; and so forth. Rather, the quality manager or technologist with limited time and lacking expertise in the computer and data analysis sciences usually applies prepackaged KPI analysis tools which are insufficient to isolate and identify specific work product-limiting factors from amongst these many potential, and intimately interrelated, factors.

The following discloses certain improvements to overcome these problems and others.

### SUMMARY

In one aspect, a non-transitory computer readable medium stores instructions readable and executable by at least one electronic processor to provide statistical analysis on one or more radiology databases in conjunction with a workstation having a display device and at least one user input device. The instructions include: instructions readable and executable by the at least one electronic processor to provide a plurality of different analysis services for selecting and processing exam data stored in the one or more radiology databases; and user guidance instructions readable and executable by the at least one electronic processor to guide a user in creating and executing a workflow by providing a graphical user interface (GUI) on the workstation having menus and/or GUI dialogs for user selection, configuration, and execution of an ordered sequence of analysis services and providing data persistence between the analysis services of the ordered sequence of analysis services.

In another aspect, a statistical analysis method on exam data stored in one or more radiology databases includes: retrieving, from the one or more radiology databases, exam data for an exam cohort; providing a GUI showing key performance indicators (KPIs) for the exam data; providing, on the GUI, one or more GUI dialogs allowing a user to define or modify at least one KPI; and updating the KPIs shown on the GUI to include the at least one defined or updated KPI.

In another aspect, an apparatus is configured to provide statistical analysis on one or more radiology databases. The apparatus includes a workstation with: at least one user input device; a display device; and at least one electronic processor programmed to: retrieve, from the one or more radiology databases, exam data for an exam cohort; provide a GUI showing KPIs for the exam data on the display device; provide, on the GUI, one or more GUI dialogs allowing a user to define or modify at least one KPI; update the KPIs shown on the GUI to include the at least one defined or updated KPI.

One advantage resides in providing an apparatus suggesting different analysis workflows for a user analyzing a radiology database.

Another advantage resides in providing an apparatus for a user to guide the user through a series of analysis workflow steps.

Another advantage resides in providing an apparatus for a user to provide the user with interactive decision points where users can provide additional filters or parameters to inform the next step of the workflow.

Another advantage resides in enabling a radiologist to apply their own knowledge to an analysis of a radiology report.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for analyzing radiology reports in accordance with the present disclosure.

FIG. 2 shows exemplary flow chart operations performed by the apparatus of FIG. 1.

FIG. 3 diagrammatically illustrates an example output generated by the apparatus of FIG. 1.

### DETAILED DESCRIPTION

This following relates to an apparatus providing a dashboard or other graphical user interface (GUI) that allows a radiology department quality manager or other analyst to perform various analyses on images stored in a Picture Archiving and Communication System (PACS), possibly in relation to other information such as patient demographic information from a Radiology Information System (RIS) database or radiology findings stored in the PACS.

Users of such a dashboard are likely to be radiology department managers or the like who may have medical/radiology expertise, but are unlikely to have a background in statistical data analysis, or the time to develop complex statistical analyses of key performance indicators (KPIs).

To service such users, the disclosed systems and methods provide an assistive analysis/insight engine. To this end, various component analysis services are provided. For example, an analysis service may be provided to define a cohort in terms of features such as imaging modality, imaged anatomy, patient demographic features, reason for examination, examination date range, et cetera. Another analysis service may be provided to randomly draw a sample population of a specified size from a defined cohort. Analysis services may be provided to construct various visualizations of specified features of a sample population.

Furthermore, the dashboard provides a set of GUI dialog screens for interfacing the user with these various services. For example, a GUI dialog screen may be provided with radial buttons, dropdown lists, and/or other GUI dialog boxes via which the user may select the features defining the cohort. Further GUI dialog screens may be provided for selecting the definition of a KPI, for selecting the visualization(s) to display, and so forth. GUI dialog screens are also provided to present analysis results, e.g. to display visualizations generated by the visualization services.

In some embodiments disclosed herein, an analysis workflow repository is provided. As a user constructs a workflow by working through the various GUI dialog screens, the resulting workflow is stored and can be saved to the repository under a suitably identifying name. Subsequently, the user (or, in some embodiments, any user with access to the repository) can retrieve the workflow and rerun it as-is, or can modify the retrieved workflow by adjusting parameters via the GUI dialog screens (which initially have values populated according to the retrieved workflow) before running it.

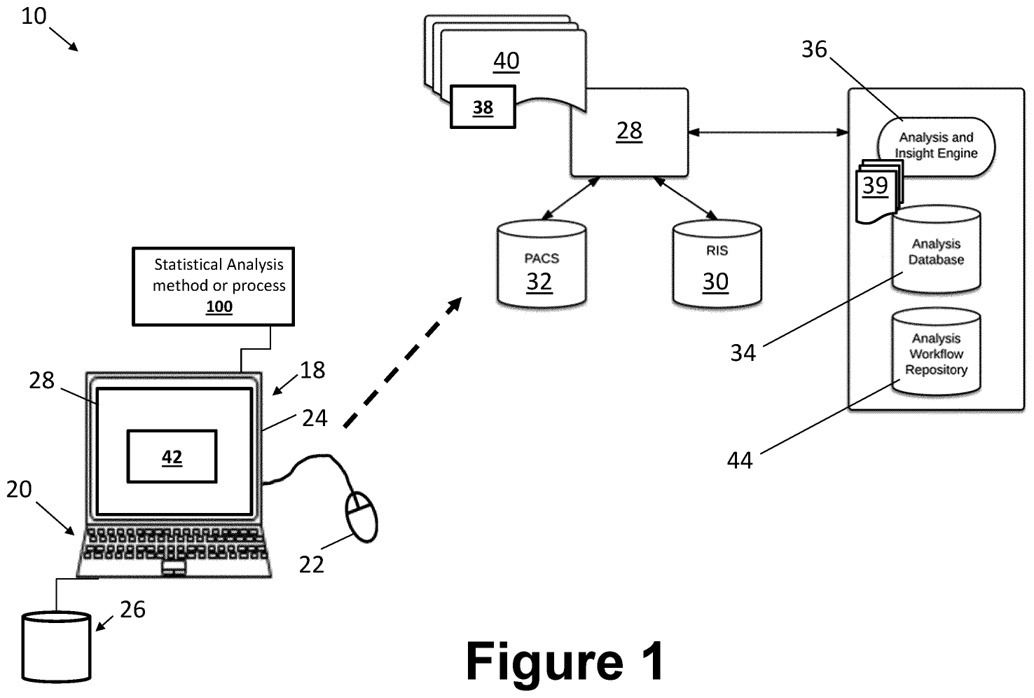

With reference to FIG. 1, an illustrative apparatus 10 for performing a statistical analysis on radiological data is shown. The apparatus 10 includes an electronic processing device 18, such as a workstation computer, or more generally a computer. The workstation 18 may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The workstation 18 is also in communication with one or more radiology databases, such as a RIS 30 and a PACS 32 (among others, such as an EMR, EHR and so forth). The workstation 18 is configured to retrieve information about the radiology examination (e.g., from the RIS), and/or images acquired during the examination (e.g., from the PACS) to perform a statistical analysis of the data stored in the RIS 30 and the PACS 32. Optionally, the workstation 18 is further configured to retrieve exam data.

The non-transitory computer readable medium 26 is configured to store instructions are readable and executable by the at least one electronic processor 20 of the workstation 18 to perform disclosed operations to analyze the data from the RIS 30 and the PACS 32. To do so, the non-transitory computer readable medium 26 can include analysis database 34 that stores device utilization data, log file analysis, clinical metrics, automated image quality assessments, reports, and images (which can be references or copies of files stored remotely in the RIS 30 and/or the PACS 32). This data, along with data from the RIS 30 and/or the PACS 32, can be analyzed by an analysis and insight engine 36 implemented in the at least one electronic processor 20. The analysis and insight engine 36 is configured to create dynamic data workflows 38 made up of various combinations of analysis services 39 with various user-defined configurations as guided by the analysis and insight engine 36. Moreover, the non-transitory computer readable medium 26 is configured to store a plurality of GUI dialogs 40 (e.g., a pop-up window, or a window comprising the entire screen of the display device 24) corresponding with workflows 38 for display on the display device 24 via the GUI 28. The GUI dialogs 40 can display visualizations 42 of the exam cohort and/or the KPIs for the cohort. The non-transitory computer readable medium 26 also includes an analysis workflow repository 44 configured to store workflows and reports generated by the user.

In some embodiments, the instructions include instructions to provide the plurality of different analysis services 39 executable by the analysis and insight engine 36 for selecting and processing exam data stored in the RIS 30 and/or the PACS 32. The analysis services 39 can include, for example, a cohort definition service for defining an exam cohort, an exam data retrieval service for retrieving exam data for an exam cohort from the RIS 30 and/or the PACS 32 (in which the exam cohort is a predefined exam cohort or an exam cohort defined by the cohort definition service), a key performance indicators (KPI) definition service for defining one or more KPIs, a KPI computation service for computing at least one KPI on exam data retrieved by the exam data retrieval service wherein the at least one KPI includes at least one predefined KPI and/or at least one KPI defined by the KPI definition service, and a presentation service for displaying, on the display device 24 of the workstation, a summary of exam data retrieved by the exam data retrieval service including at least one KPI computed by the KPI computation service.

In other embodiments, the instructions can include user guidance instructions readable and executable by the analysis and insight engine 36 to guide a user in creating and executing one or more workflows 38 by providing the GUI 28 on the workstation including the menus and/or GUI dialogs 40 for user selection, configuration, and execution of an ordered sequence of analysis services (chosen by the user from the plurality of analysis services 39 under guidance of the analysis and insight engine 36) and providing data persistence between the analysis services of the ordered sequence of analysis services. (As used herein, the term “data persistence” refers to data retrieved is “persisted”, i.e. stored at least in RAM, and then loaded into the KPI computation service; likewise, a KPI defined by the KPI definition service is persisted and loaded into the KPI computation service, and so forth).

In some examples, the user guidance instructions guide the user to create and execute the workflow in which the ordered sequence of analysis services includes at least the cohort definition service, the KPI computation service, and the presentation service executed in that order. That is, the order of operations includes the GUI 28 guiding the user to creating the cohort, computing the KPIs for that cohort, and displaying the KPIs on the GUI. In other examples, the user guidance instructions guide the user to create and execute the workflow 38 in which the ordered sequence of analysis services further includes execution of the KPI definition service before execution of the KPI computation service (and, optionally, defining the KPI before computing the KPI).

In further embodiments, the instructions further include workflow archiving instructions readable and executable by the analysis and insight engine 36 to save a workflow 38 to the workflow repository 44, retrieve a workflow from the workflow repository, and execute the retrieved workflow. For example, the workflow archiving instructions can invoke the user guidance instructions to guide a user in modifying the retrieved workflow prior to executing the retrieved workflow 38.

The apparatus 10 is configured as described above to perform a statistical analysis method or process 100 for analyzing data stored in the RIS 30 and/or the PACS 32. The non-transitory storage medium 26 stores instructions which are readable and executable by the at least one electronic processor 20 of the workstation 18 to perform disclosed operations including performing the statistical analysis method or process 100. In some examples, the method 100 may be performed at least in part by cloud processing.

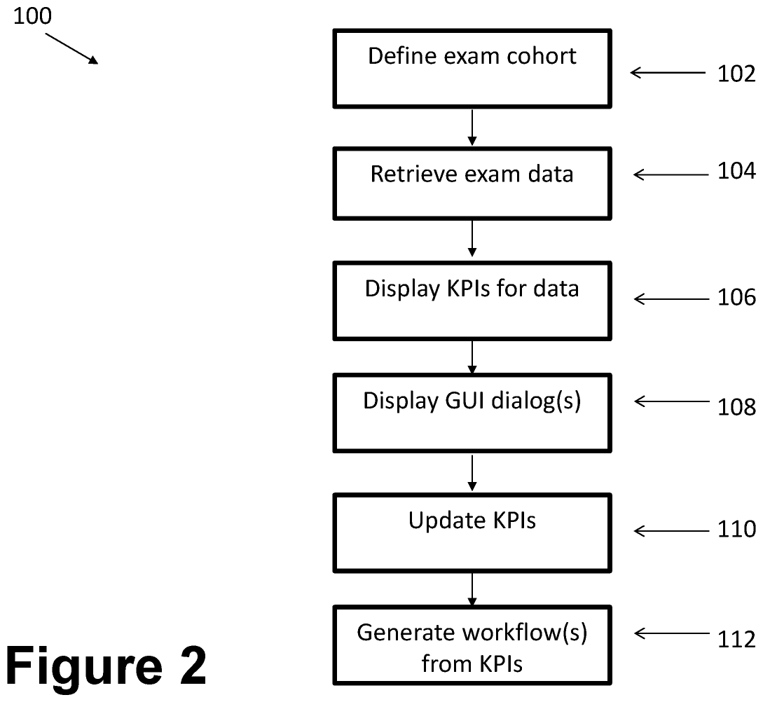

With reference to FIG. 2, and with continuing reference to FIG. 1, an illustrative embodiment of the statistical analysis 100 is diagrammatically shown as a flowchart. At an operation 102, one or more GUI dialogs 40 are provided on the GUI 28 displayed on the display device 24 for defining an exam cohort in terms of clinical features including one or more of an imaging modality, an imaged anatomy of one or more patients, patient demographic features, a reason for examination, and examination date range.

At an operation 104, exam data for the exam cohort is retrieved from the RIS 30 and/or the PACS 32. At an operation 106, KPIs for the exam cohort data is displayed on the GUI 28. At an operation 108, one or more GUI dialogs 40 are displayed on the GUI to allow the user to define or modify at least one of the displayed KPIs. At an operation 110, the KPIs are updated on the GUI to include the defined or updated KPI(s).

In some embodiments, after the GUI 28 is provided to show the KPIs, a GUI dialog 40 can be provided by which the user can modify the clinical features that define the cohort. The exam cohort can be updated on the GUI 28 based on the modified clinical features.

In other embodiments, after the GUI 28 is provided to show the KPIs, a GUI dialog 40 can be provided by which the visualizations 42 of the exam cohort and/or the KPIs can be displayed. The exam cohort can be updated on the GUI 28 based on the modified clinical features. The visualizations 42 can be updated based on one or more user inputs from the user via at least one user input device 22.

In some embodiments, the method 100 can also include an operation 112, in which one or more workflows 38 are generated based on user inputs defining the defined or modified KPIs, and/or the exam cohort. The workflows 38 can then be stored in the repository 44. In some examples, one or more workflows 38 can be retrieved from the repository 44, and executed to re-create or re-generate the GUI 28 showing the KPIs for the exam data.

The following is an example of the method 100. A radiology modality quality manager launches the radiology quality dashboard GUI 28 and is provided with a default view of the KPIs for their department and modality. The manager selects a KPI on the GUI 28 to explore further, based on a specific modality across various anatomies, reasons for exam, and patient demographics. The analysis and insight engine 36 provides a graphical summary of each of the data types that has contributed to the KPI on the GUI 28, as well as a selection of previous analysis workflows 38 that have been previously executed against either the same KPI or any of the data types that contribute to the KPI. The radiology modality quality manager can either select a previously executed workflow 38 to explore the data or create a new workflow based on their own parameters and filters. The analysis and insight engine 36 leads the quality manager through each step of creating a new workflow by data type, requesting parameters or filters to apply at each step. Once the workflow 38 has been completed and the analysis finalized, the manager can save and name the workflow as well as a final report. The final view of the analysis is interactive and any of the filters or parameters applied during the workflow can be adjusted in real time to explore the data further.

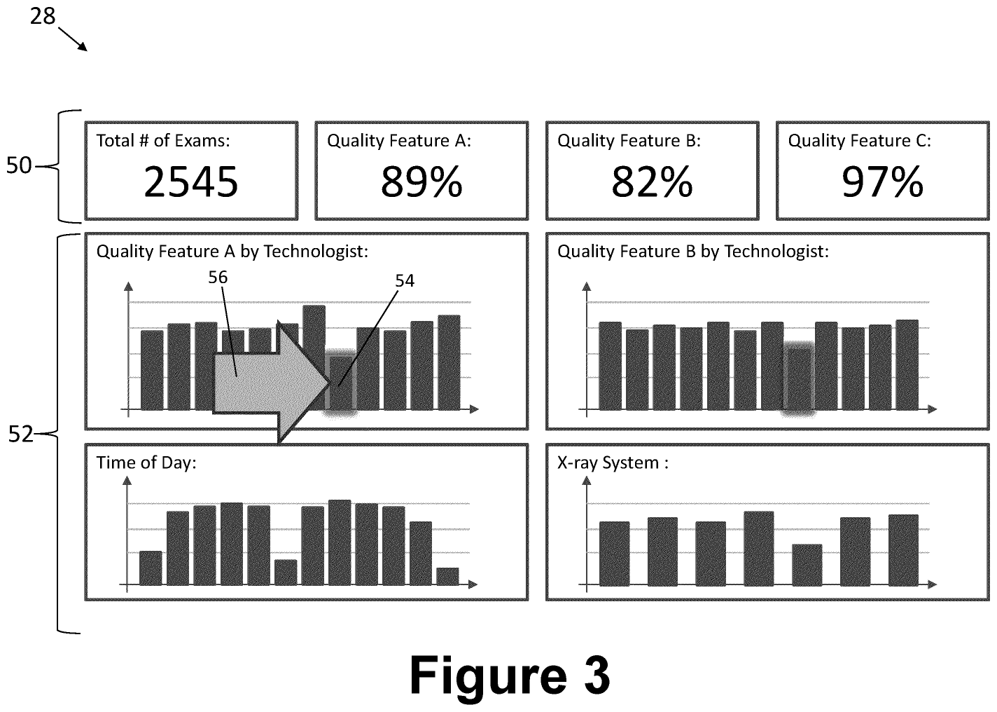

With reference to FIG. 3, an illustrative example of a visualization 42 provided on the GUI 28 is shown. In the visualization 42, a set of quantitative indicators 50 shows a quick overview over the data in the non-transitory computer readable medium 26 and/or the database 30. As shown in FIG. 2, four quantitative indicators 50 are shown, and can include, for example, a total number of reports (e.g., 2545 exams), a percentage of those reports having a quality feature A (e.g., 89%), a percentage of those reports having a quality feature B (e.g., 82%), a percentage of those reports having a quality feature C (e.g., 97%), and so forth. The visualization 42 also includes one or more plots 52 representing features of the detected quality features. For example, FIG. 2 shows four plots 52 as bar graphs: (i) a bar graphs showing the number of times quality feature A is detected per radiologist; (ii) a bar graphs showing the number of times quality feature B is detected per radiologist; (iii) a bar graphs showing a time of day the exams were acquired; and (iv) a bar graphs showing a number of exams per imaging system (e.g., an X-ray system). In addition, one or more annotating features 54, 56 can be included in the visualization 42 to highlight outliers in the plots 52. For example, an outlier bar in the one of the bar graphs can be highlighted in a color (designated as “54”), and further be identified with a symbol, such as an arrow designated as “56”. These are merely examples, and should not be construed as limiting.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by at least one electronic processor to provide statistical analysis on one or more radiology databases in conjunction with a workstation having a display device and at least one user input device, the instructions comprising:
instructions readable and executable by the at least one electronic processor to provide a plurality of different analysis services for selecting and processing exam data stored in the one or more radiology databases; and
user guidance instructions readable and executable by the at least one electronic processor to guide a user in creating and executing a workflow by providing a graphical user interface (GUI) on the workstation having menus and/or GUI dialogs for user selection, configuration, and execution of an ordered sequence of analysis services and providing data persistence between the analysis services of the ordered sequence of analysis services.

2. The non-transitory computer readable medium of claim 1, wherein the plurality of analysis services include at least:
a cohort definition service for defining an exam cohort,
an exam data retrieval service for retrieving exam data for an exam cohort from the one or more radiology databases wherein the exam cohort is a predefined exam cohort or an exam cohort defined by the cohort definition service,
a key performance indicators (KPI) definition service for defining one or more KPIs,
a KPI computation service for computing at least one KPI on exam data retrieved by the exam data retrieval service wherein the at least one KPI includes at least one predefined KPI and/or at least one KPI defined by the KPI definition service, and
a presentation service for displaying, on the display device (24) of the workstation, a summary of exam data retrieved by the exam data retrieval service including at least one KPI computed by the KPI computation service.

3. The non-transitory computer readable medium of claim 2, wherein the user guidance instructions guide the user to create and execute the workflow in which the ordered sequence of analysis services includes at least the cohort definition service, the KPI computation service, and the presentation service executed in that order.

4. The non-transitory computer readable medium of claim 3, wherein the user guidance instructions guide the user to create and execute the workflow in which the ordered sequence of analysis services further includes execution of the KPI definition service before execution of the KPI computation service.

5. The non-transitory computer readable medium of claim 1, wherein the instructions further comprise:
workflow archiving instructions readable and executable by the at least one electronic processor to:
save a workflow to a workflow repository,
retrieve a workflow from the workflow repository, and
execute the retrieved workflow.

6. The non-transitory computer readable medium of claim 5, wherein the workflow archiving instructions are further readable and executable by the at least one electronic processor to invoke the user guidance instructions to guide a user in modifying the retrieved workflow prior to executing the retrieved workflow.

7. A statistical analysis method on exam data stored in one or more radiology databases, the method comprising:
retrieving, from the one or more radiology databases, exam data for an exam cohort;
providing a graphical user interface (GUI) showing key performance indicators (KPIs) for the exam data;
providing, on the GUI, one or more GUI dialogs allowing a user to define or modify at least one KPI;
updating the KPIs shown on the GUI to include the at least one defined or updated KPI.

8. The method of claim 7, further including:
prior to the retrieving, providing, on the GUI, one or more GUI dialogs for defining the exam cohort in terms of clinical features including one or more of an imaging modality, an imaged anatomy of one or more patients, patient demographic features, a reason for examination, and examination date range.

9. The method of claim 7, wherein the at least one radiology database includes at least one of a Radiology Information System (RIS) database and a Picture Archiving and Communication System (PACS) database.

10. The method of claim 7, wherein providing the one or more GUI dialogs includes:
after providing the GUI showing the KPIs for the exam data, providing a GUI dialog by which the user modifies the clinical features defining the cohort; and
updating the exam cohort based on the modified clinical features.

11. The method of any one of claims 7, wherein providing of the GUI showing the KPIs includes:
providing a GUI dialog displaying a selection of statistical visualizations of the KPIs and/or the exam cohort;
updating the statistical visualizations based on one or more user inputs from the user via at least one user input device.

12. The method of claim 7, further including:
generating one or more workflows based on user inputs defining the defined or modified KPIs, and/or the exam cohort; and
storing the generated one or more workflows in a workflow repository.

13. The method of claim 12, further including:
retrieving the generated one or more workflows stored in the repository; and
executing the retrieved one or more workflows to re-create the GUI (28) showing the KPIs for the exam data.

14. An apparatus configured to provide statistical analysis on one or more radiology databases, the apparatus comprising a workstation including:
at least one user input device;
a display device; and
at least one electronic processor programmed to:
retrieve, from the one or more radiology databases, exam data for an exam cohort;
provide a graphical user interface (GUI) showing key performance indicators (KPIs) for the exam data on the display device;
provide, on the GUI, one or more GUI dialogs allowing a user to define or modify at least one KPI; and
update the KPIs shown on the GUI to include the at least one defined or updated KPI.

15. The apparatus of claim 14, wherein the at least one electronic processor is further programmed to:
generate one or more workflows based on user inputs defining the defined or modified KPIs, and/or the exam cohort; and
store the generated one or more workflows in a workflow repository.

16. The apparatus of claim 15, wherein the at least one electronic processor is further programmed to:
retrieve the generated one or more workflows stored in the repository; and
execute the retrieved one or more workflows to re-create the GUI showing the KPIs for the exam data.

17. The apparatus of claim 15, wherein the at least one electronic processor (20) is further programmed to:
prior to the retrieving, provide, on the GUI, one or more GUI dialogs for defining the exam cohort in terms of clinical features including one or more of an imaging modality, an imaged anatomy of one or more patients, patient demographic features, a reason for examination, and examination date range.

18. The apparatus of claim 15, wherein the at least one electronic processor is further programmed to:
after providing the GUI showing the KPIs for the exam data, provide a GUI dialog by which the user modifies the clinical features defining the cohort; and
update the exam cohort based on the modified clinical features.

19. The apparatus of claim 15, wherein the at least one electronic processor is further programmed to:
provide a GUI dialog displaying a selection of statistical visualizations of the KPIs and/or the exam cohort;
update the statistical visualizations based on one or more user inputs from the user via at least one user input device.

20. The apparatus of claim 15, wherein the at least one radiology database includes at least one of a Radiology Information System (RIS) database and a Picture Archiving and Communication System (PACS) database.
